(19) United States
(12) Patent Application Publication
(10) Pub. No.: US 2015/0297334 A1
(43) Pub. Date: Oct. 22, 2015

(54) Implantable Hernia Patch

(71) Applicant: Alfredo FERNANDEZ, Tampa, FL (US)

(72) Inventor: Alfredo Fernandez, Tampa, FL (US)

(21) Appl. No.: 14/649,450

(22) PCT Filed: Dec. 3, 2013
(86) PCT No.: PCT/US13/72845

§ 371 Date: Jun. 3, 2015

Related U.S. Application Data
(60) Provisional application No. 61/733,155, filed on Dec. 4, 2012.

(51) Int. Cl. A61F 2/00

(52) U.S. Cl. CPC A61F 2/0063; A61F 2002/0072; A61F 2002/009

(57) ABSTRACT

An implantable hernia repair patch comprising a first portion in the form of a tubular surgical plastic mesh having a proximal and distal end and a second portion having the form of a planar sheet of surgical plastic mesh having a distal surface and a proximal surface. In some embodiments the first portion and the second portion are a single piece of surgical mesh. In other embodiments the first portion is attached the second portion by to form the patch. The first portion can be connected to the second portion by stitching, bonding, adhesives, or staples.

### RELATED APPLICATIONS

This application claims the benefit of priority to U.S. Provisional Application No. 61/733,155, entitled Implantable Hernia Patch, and filed on Dec. 4, 2012, the contents of which are incorporated herein in their entirety.

### TECHNICAL FIELD

This invention relates to laparoscopic hernia repair. More particularly, it refers to a flared mesh patch to repair an inguinal hernia during laparoscopic procedures and methods of using the same.

### BACKGROUND

The traditional inguinal approach to hernia repair results in recurrence ranging from seven to twenty-one percent for primary repair and up to eighteen percent after repair of recurrent hernias. In addition, recovery periods associated with herniorrhaphy can be lengthy and painful, sometimes lasting from three to six weeks. See the Journal of Laparoendoscopic Surgery, Volume 1, No. 5, p. 269, 1991, Mary Ann Liebert, Inc., publishers. In seeking to improve on these results and reduce recovery periods associated with herniorrhaphy, surgeons have been carrying out laparoscopic hernia repair employing laser surgery techniques. See the Journal of Laparoendoscopic Surgery, Vol. 1, No. 1, 1990, pages 41-45; and Contemporary Surgery, October 1991, Vol. 39, No. 14, pages 15-19.

While it appears that laparoscopic hernia repair techniques have been successful, the insertion of polypropylene mesh into the opening in the inguinal region causing the hernia has sometimes resulted in bulging of the inguinal region. This could be caused by movement of the rolled up surgical mesh inserted into the hernia opening. A more perfect prosthetic mesh patch that can be delivered to the hernia site by laparoscopic techniques is needed to reduce side effects and recurrences from laparoscopic hernia repair surgery.

### SUMMARY

Implementations of the present invention are directed to an improved patch for use in laparoscopic hernia repair surgery and methods of using the same.

In an example implementation the patch is made by rolling up a first sheet of a surgical plastic mesh, maintaining the rolled up configuration by at least two circumferential bands of cat gut, cutting longitudinal slits in one end of the rolled up mesh and flaring out the multiple flaps formed by the slits. A second planar sheet of surgical plastic mesh is then stitched to the flared out flaps. The flaps and planar sheet are then compressed together as a longitudinal extension of the first rolled up sheet and inserted into a cylindrical plastic delivery tube. The tube is inserted into the abdomen via a trocar and the patch is pushed out by a plunger into the hernia opening. The second planar sheet is stapled to the tissue adjacent the hernia opening with a stapling gun.

In another example implementation the patch comprises a first portion in the form of a tubular surgical plastic mesh having a proximal and distal end and a second portion having the form of a planar sheet of surgical plastic mesh having a distal surface and a proximal surface. In some embodiments the first portion and the second portion are a single piece of surgical mesh. In other embodiments the first portion is attached the second portion by to form the patch. The first portion can be connected to the second portion by stitching, bonding, adhesives, or staples.

In yet another example implementation the patch comprises a first portion having a tubular form with a proximal end and a distal end and an inner surface and an outer surface. The patch further comprises a planar sheet having a distal surface and a proximal surface. The first portion is connected to the second portion such that at the distal end of the first portion the inner surface of the first portion seamlessly merges to the distal surface of the second portion and the outer surface of the first portion seamlessly merges to the proximal surface of the second portion.

In still a further example of the present invention, the distal surface of the planar sheet second portion comprises an outer layer to resist adhesion of the patch to bodily tissue. In one embodiment the proximal portion of the planar sheet second portion does not include an adhesion resistant outer layer. In another embodiment the distal surface and the proximal surface of the second portion and the inner surface of and the outer surface of the first portion include an adhesion resistant outer layer. The adhesion resistant outer layer may comprise ePTFE or PTFE.

In certain embodiments the first and second portions may comprise any surgical plastic mesh material suitable for use in hernia operations, including PROLENE, sold by Ethicon, Inc., or a polypropylene or a polytetrafluoroethylene. The planar sheet second portion may be in the form of an oval, a circle, a square, a rectangle, a polygon, a tetrahedron, and an irregular form. The first portion, the second portion, or the first and second portion may include a shape memory feature, and/or may be resistant to deformation once unfolded and/or implanted.

### DESCRIPTION OF DRAWINGS

The invention may be best understood by those having ordinary skill in the art by reference to the following detailed description when considered in conjunction with the accompanying drawings in which:

FIG. 1A is a top plan view of a piece of surgical plastic mesh being rolled into a cylindrical shape.

FIG. 1B is an elevation view of the rolled up surgical mesh of FIG. 1A with slits at one end.

FIG. 2 is a perspective view of the patch of this invention.

FIG. 3 is a perspective view of a rolled up patch being inserted into a delivery tube.

FIG. 4 is a perspective view partially in phantom showing the patch being pushed out of the delivery tube.

FIG. 5 is a frontal view of a patient undergoing laparoscopic hernia repair surgery.

FIG. 6 is a section view along lines 6-6 of FIG. 5 through the abdomen of a patient with a hernia visible.

FIG. 7 is a section view along lines 6-6 of FIG. 5 adding a showing of the patch being inserted into the opening of the hernia.

FIG. 8 is a section view along lines 6-6 of FIG. 5 adding a showing of the patch in place over the opening of the hernia.

FIG. 9 is a perspective view of an alternate embodiment of the present invention.

### DETAILED DESCRIPTION

Throughout the following detailed description, the same reference numerals refer to the same elements in all figures.

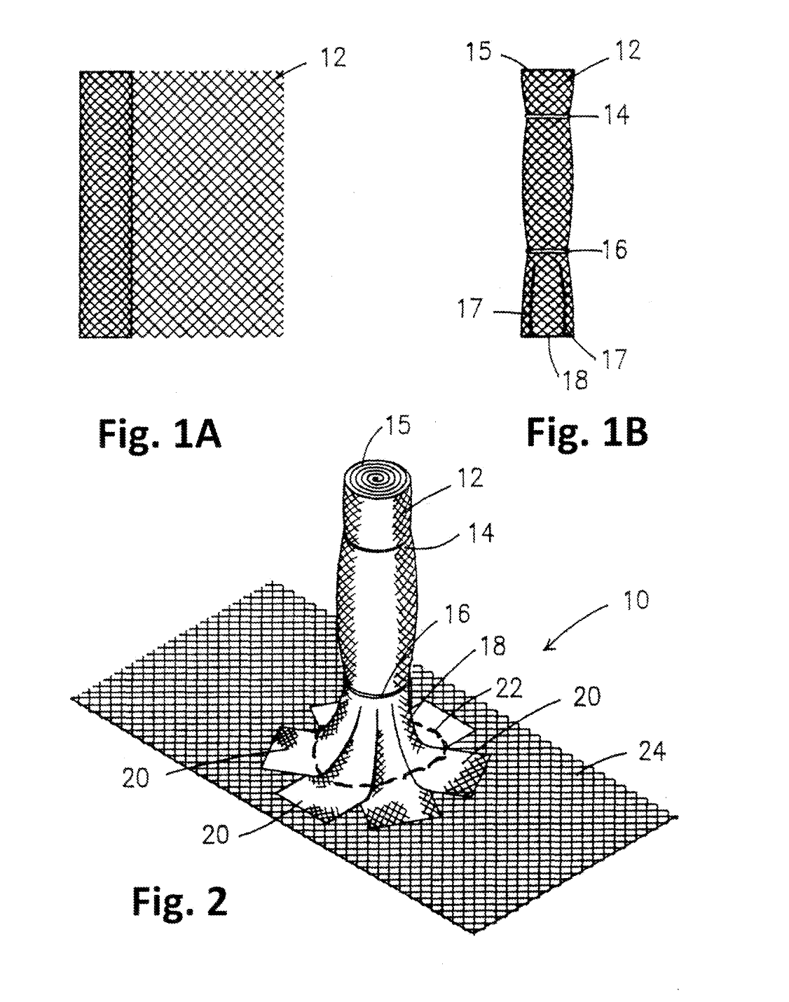

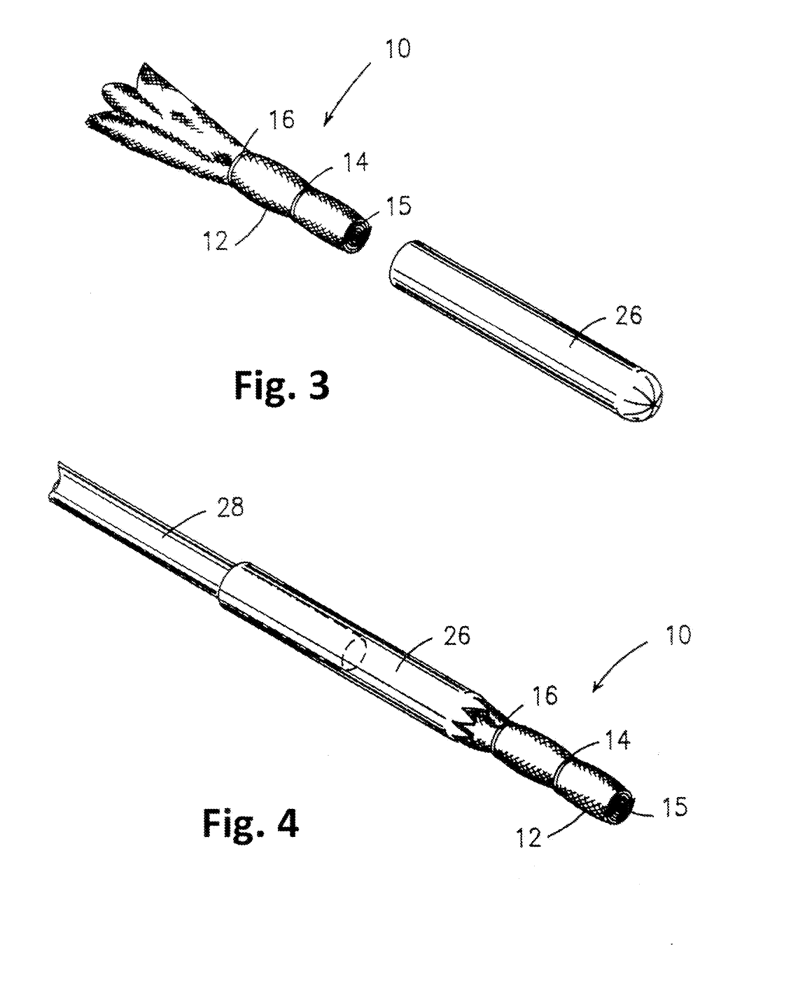

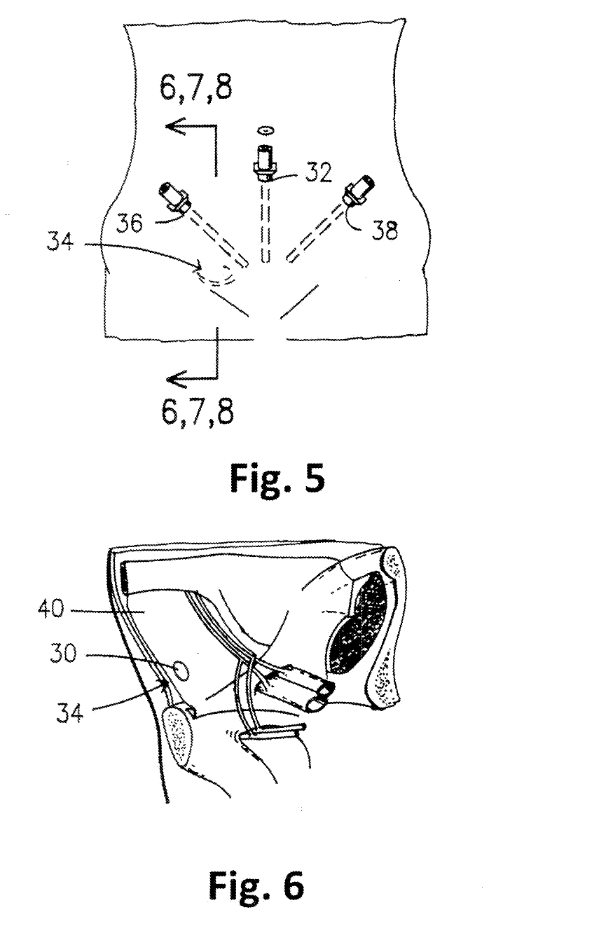

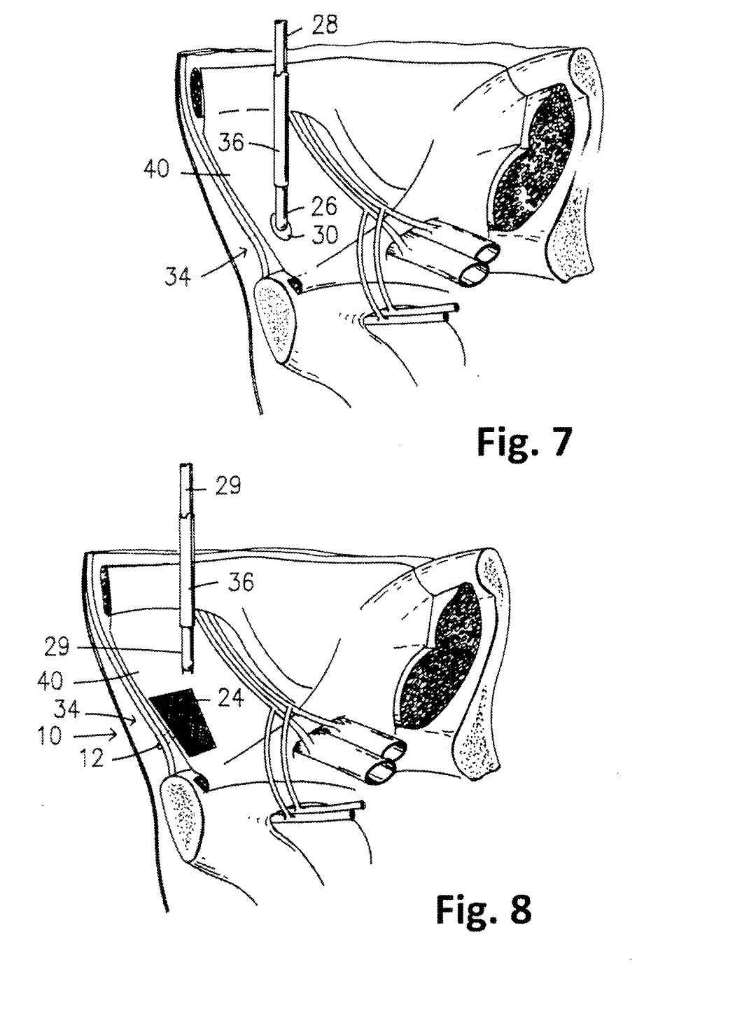

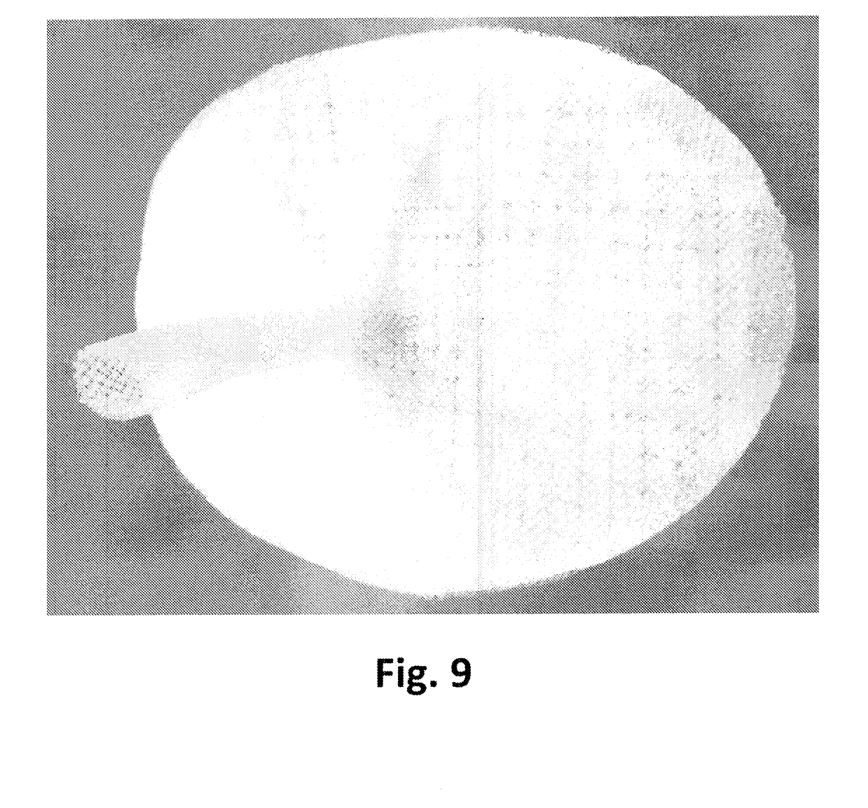

The prosthetic mesh patch 10 of the invention shown in FIG. 2 has a first rolled up surgical plastic mesh portion 12 tied together by cat gut 14 near a first end 15 and a cat gut 16 about midway to two-thirds down the rolled up surgical plastic mesh 12. Approximately five scissor slits 17 are made in the second end 18 of the rolled surgical plastic 12 so that six flared flaps 20 are produced. The flaps 20 are stitched 22 to a flat sheet 24 of surgical mesh. Thereafter, the patch 10 is rolled up as shown in FIG. 3 and inserted into a plastic carrier 26. An elongated plunger 28 as shown in FIG. 4 is used to push out the patch 10 from its carrier 26 into the opening 30 of the hernia.

As illustrated in FIG. 2, flat sheet 24 is of a generally rectangular shape approximately 120 mm in height and 90 mm in width. In some embodiments, flat sheet 24 may be of a generally circular, ovular, square, or polygonal shape. In some embodiments, the generate height and width of flat sheet 24 may be larger or smaller, depending on the physical constraints of the region in which the patch will be installed. In some embodiments, the dimensions and shape of flat sheet 24 may be varied to be more easily inserted into the plastic carrier 26. In some embodiments, flat sheet 24 is not completely flat on a single plane, but rather of a three dimensional shape, such that it may fit more precisely against a non-flat surface. In such embodiments, flat sheet 24 may be of a generally conical, hemispherical, ellipsoidal, polyhedral, or irregular shape.

As illustrated in FIGS. 1 and 2, plastic mesh 12 is rolled up several times to create a generally tubular shape. In some embodiments, plastic mesh 12 is rolled up fewer times while maintaining generally the same dimensions, creating a semi-hollow tubular shape that is more easily deformed for insertion into the plastic carrier 26.

In some embodiments, plastic mesh portion 12 may be fabricated in a three-dimensional tubular design, such that cat gut 14 or other similar securing method is not required to maintain its generally tubular shape. In some embodiments, plastic mesh portion 12 may be fabricated in a three-dimensional design that is generally tubular in shape, but with asymmetrical dimensional variations, such that it may fit into an irregularly shaped orifice. Example shapes include a funnel, a bent tube, or a tube with a bulging central area, among others.

In some embodiments, plastic mesh 12 may be attached to flat sheet 24 using means other than stitched flaps. In some embodiments, plastic mesh 12 may be attached to flat sheet 24 using biocompatible adhesives, staples, or other fastening techniques and methods. In some embodiments, plastic mesh 12 and flat sheet 24 may of a single three-dimensional design, such that no fastening is required.

The patch 10 is used for repeating inguinal hernias. In repairing such a hernia, a patient is brought into an operating room and placed in the supine position. General endotracheal anesthesia is induced and the abdomen, groin and genitalia are prepped and draped in a sterile fashion using betadine solution. The patient is placed in a Trendelenburg position. A small vertical incision is made in the lower edge of the umbilicus and a Verres needle is inserted into the abdomen and a pneumoperitoneum of approximately three liters is carried out. A ten millimeter trocar 32 is then placed through the umbilicus and a laparoscope is threaded through the trocar 32. The abdomen is inspected and the repair site 34 is visually observed. Usually there is a small sac associated with the hernia opening. A five millimeter trocar 36 is then inserted in the right abdomen and a twelve millimeter trocar 38 in the left abdomen parallel with the umbilicus. The dissecting trocars are inserted into the abdomen and the hernia sac is dissected out using a contact YAG laser to excise the sac. Normally, no bleeding is encountered. The patch 10 is backloaded into a twelve millimeter sleeve of an endopath stapler 29 and inserted into the abdomen through trocar 36. Alternatively, the patch is pushed out of carrier 26 by plunger 28 through the trocar 36. The patch 10 is inserted into the opening 30 so that the rolled up portion 12 is completely inserted into the opening. The flared portion 20 with the second plastic mesh sheet 24 is then stapled in place to adjacent tissue by the endopath stapler 29. The incisions are repaired and the patient is then sent for recovery.

The stapler 29 used for stapling the patch to the adjacent tissue is an endoscopic tissue repair stapling gun, such as, the ENDOPATH ES Endoscope sold by Ethicon, Inc., a division of Johnson & Johnson Co. Such a stapler is designed for use with a twelve millimeter disposable surgical trocar.

The surgical plastic mesh employed to make the patch of this invention can be any of the surgical plastic meshes suitable for use in hernia operations, such as, PROLENE, sold by Ethicon, Inc. Generally, the plastic mesh is either a polypropylene or a polytetrafluoroethylene.

Specific portions of the present invention may be made of differing materials in order to vary the interaction between the present invention and the tissue upon it is implanted. In some embodiments, the plastic mesh portion 12 may be made of a polypropylene, such that a fibroblastic reaction is induced upon tissue that is contacted by the plastic mesh portion 12. Such a fibroblastic reaction may form a strong fibrous wall of tissue around and through the plastic mesh, therefore creating a bond between the mesh portion 12 and the tissue in order to further strengthen the repair.

In some embodiments, the flat sheet 24 may be made of a polypropylene in order to induce a similar fibroblastic reaction between the flat sheet 24 and the underlying tissue. In some embodiments, the flat sheet 24 may be made of polytetrafluoroethylene, a material with a low coefficient of friction and known to elicit minimal fibroblastic response upon interaction with tissue. In such embodiments, tissue contacted by flat sheet 24 will not form a fibrous wall around and through the flat sheet 24, and no bond will be formed.

In some embodiments, flat sheet 24 may be made of polypropylene on one face and polytetrafluoroethylene on the other face, such that tissue coming in contact with one face of the flat sheet 24 will become bonded through fibroblastic reaction, but tissue coming in contact with the other face of the flat sheet 24 will not. These embodiments may be used to selectively bond tissue to the flat sheet 24 to structurally strengthen the region of repair, while ensuring that vital tissue and organs, such as the bowels, are not undesirably bonded to the flat sheet 24. In some embodiments, plastic mesh patch 10 made be made of polypropylene, while a second patch (not shown) made of polytetrafluoroethylene may be attached to one face of flat sheet 24, in order to provide physical separation between flat sheet 24 and vital tissue and organs. The second patch (not shown) may be attached to flat sheet 24 using biologically compatible adhesive, staples, stitches, or other fastening techniques and methods.

The stitches 22 used to keep the second plastic mesh 24 in place over the flaps 20 are also either a polypropylene or polytetrafluoroethylene thread commonly used in surgical sutures. The size of the plastic surgical mesh used in this invention is usually a two and one-half by four inch sheet of expanded polytetrafluoroethylene or polypropylene and such a sheet can be used for either the first rolled up mesh 12 or the second flat mesh 24. The mesh 24 has to be large enough to overlap the opening of the hernia and provide adequate room for stapling into the adjacent peritoneum 40.

Modification of the materials used in the patch of this invention can be made depending upon the availability of new types of surgical mesh and improvements in the stitching or stapling materials.

Due to the ease by which the present invention may be introduced into the human body, manipulated, and attached using existing laparoscopic surgical techniques, the present invention is ideal for use in endoscopic hernia repair. In particular, the present in invention may be used to repair indirect hernias, small direct hernias with well-defined margins and an otherwise strong abdominal floor, or recurrent inguinal hernias with defects in an otherwise strong abdominal floor.

What is claimed is:

1. A surgical flared patch for use in laparoscopic hernia repair comprising:
a first surgical mesh of generally planar shape; and
a second surgical mesh of generally tubular shape, longitudinally attached to the first surgical mesh;
wherein the first surgical mesh and second surgical mesh are capable of being compressed together in a generally tubular shape such that it can be inserted into a cylindrical delivery tube.

2. The surgical patch of claim 1 wherein the first portion and the second portion are a single piece of surgical mesh.

3. The surgical patch of claim 1 wherein the first portion is attached to the second portion by to form the patch.

4. The patch of claim 3 wherein the first portion is connected to the second portion by stitching, bonding, adhesives, or staples.

5. A surgical flared patch for use in laparoscopic hernia repair comprising:
a first portion having a substantially tubular form with a proximal end and a distal end and an inner surface and an outer surface, and
a second portion forming a substantially planar sheet having a distal surface and a proximal surface,
wherein the first portion is connected to the second portion such that at the distal end of the first portion and the inner surface of the first portion seamlessly merges to the distal surface of the second portion and the outer surface of the first portion seamlessly merges to the proximal surface of the second portion.

6. The surgical flared patch of claim 5 wherein the distal surface of the planar sheet second portion comprises an outer layer to resist adhesion of the patch to bodily tissue.

7. The patch of claim 5 wherein the proximal portion of the planar sheet second portion does not include an adhesion resistant outer layer.

8. The patch of claim 5 wherein the distal surface and the proximal surface of the second portion and the inner surface of and the outer surface of the first portion include an adhesion resistant outer layer.

9. The patch of claim 6 wherein the adhesion resistant outer layer comprises ePTFE or PTFE.

10. The patch of claim 1 further comprising an adhesion outer layer.
